(19) United States
(12) Patent Application Publication
(10) Pub. No.: US 2009/0232782 A1
(43) Pub. Date: Sep. 17, 2009

(54) Method for treating brain ischemic injury through transplantation of human umbilical mesenchymal stem cells

(75) Inventor: Yu-Show Fu, Taipei (TW)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignees: Yu-Show Fu, Taipei (TW); Henrich Cheng, Taipei (TW)

(21) Appl. No.: 12/378,259

(22) Filed: Feb. 12, 2009

Related U.S. Application Data
(60) Provisional application No. 61/069,364, filed on Mar. 14, 2008.

(51) Int. Cl. A61K 35/48

(52) U.S. Cl. 424/93.7

(57) ABSTRACT

A method for treating or preventing an ischemic brain injury or neurological damage due to ischemia in a subject includes transplanting a therapeutically effective amount of human umbilical mesenchymal stem cells (HUMSCs) obtained from Wharton's Jelly to the ischemic areas of the brain injury or the neurological damage of the subject. Recovery from neurological behavior deficits also is improved according by the method.

### CROSS-REFERENCE TO RELATED APPLICATION

This application claims the benefit under 35 U.S.C. §119(e) of U.S. Provisional Patent Application No. 61/069,364, filed Mar. 14, 2008, the entire disclosure of which is hereby incorporated by reference herein.

### FIELD OF THE INVENTION

The present invention is related to neurology and stems cells.

### BACKGROUND OF THE INVENTION

Stroke is a leading disease of death and disability, caused by obstruction or rupture of cerebral vascular vessels. The interruption of cerebral blood flow leads to neural injury and irreversible long-term sensorimotor deficits. This damage caused by energy depletion, exitotoxicity, peri-infarct depolarization, inflammation and programmed cell death (Dimagl, U., et al., Trends in Neurosciences 1999, 22: 391-397; Graham, S. H., et al., Journal of Cerebral Blood Flow and Metabolism 2001, 21: 99-109; Allan, S. M., et al., Nature Reviews Neuroscience 2001, 2: 734-744). Only a few treatment options exist despite intensive research. For example, tissue plasminogen activator (tPA) is effective for treatment of patients with ischemic stroke, but only if given within the first 3 hours (NINDS, The New England Journal of Medicine 1995, 333: 1581-1587; Bednar, M. M., et al., Stroke 1999, 30: 887-893). There is no therapy capable of restoring stroke damage completely until recently.

Treatment to promote recovery typically focuses on encouraging neuronal growth and rewiring. Growth factors are currently evaluated as therapeutics in stoke and neurodegeneration. Besides direct neurotrophic effects, they promote proliferation, survival, and differentiation of endogenous neural precursor cells. Among the growth factors with neuroregenerative potential, erythropoietin (EPO), granulocyte-colony stimulating factor (G-CSF), vascular endothelial growth factor (VEGF), epidermal growth factor (EGF), glial cell line-derived neurotrophic factor (GDNF), insulin-like growth factor-1 (IGF-1), and the stem cell factor (SCF) are all prevalent targets (Wiltrout, C., et al., Neurochemistry International 2007, 50:1028-1041).

Another potential approach to treatment for stroke recovery is the use of neural stem cells. In recent years, the transplantation of neural stem or progenitor cells—whether embryonic or adult origin—has been discussed as an alternative to the activation of endogenous stem cells residing in the brain (Lindvall, O., et al., Nature 2006, 441:1094-1096). A study showed that monkey embryonic stem (ES)-cell-derived progenitors transplanted into the brains of mice after stoke differentiated into various types of neuron and glial cell, re-established connections with target areas, and led to improved motor function (Ikeda R., et al., Neurobiol Dis. 2005, 20(1): 38-48). Finkelstein et al. also disclosed that in a stoke model, rats administered intracistemally with fetal neural stem cells plus basic fibroblast growth factor (bFGF) had better behavior performance in various behavioral tests than those without treatment (U.S. Pat. No. 6,749,850). The therapeutic efficacy of such strategies could be improved further by genetically modifying the stem cells: for example, by over-expressing a VEGF gene (Maurer, M. H., et al., Int. J. Biol. Sci. 2008, 4(1): 1-7).

### BRIEF SUMMARY OF THE INVENTION

The present invention is based on the discovery of the therapeutic effects of transplantation of HUMSCs to a subject in need of treatment of ischemic brain injury.

Accordingly, one aspect of the present invention relates to a method for treating or preventing an ischemic brain injury or neurological damage due to ischemia in a subject, the method comprising transplanting a therapeutically effective amount of human umbilical mesenchymal stem cells (HUMSCs) obtained from Wharton's Jelly to the ischemic areas of the brain injury or the neurological damage of the subject.

In another aspect, the present invention relates to a method for recovering neurological behavior deficits from an ischemic brain injury or neurological damage due to ischemia in a subject, the method comprising transplanting human umbilical mesenchymal stem cells (HUMSCs) obtained from Wharton's Jelly to the ischemic areas of brain injury or neurological damage of the subject.

The details of one or more embodiments of the invention are set forth in the description below. Other features or advantages of the present invention will be apparent from the following detailed description of several embodiments, and also from the appended claims.

### BRIEF DESCRIPTION OF THE SEVERAL VIEWS OF THE DRAWING

The patent or application file contains at least one drawing executed in color. Copies of this patent or patent application publication with color drawings will be provided by the Office upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the embodiments shown in the drawings.

In the drawings:

FIG. 1A-FIG. 1D are images showing the changes of cerebral infarct volume caused by medial cerebral artery occlusion (MCAO). FIG. 1A-FIG. 1D provide 2,3,5-triphenyltetrazolium chloride (TTC) stain images of rat brains at day 1 (FIG. 1A), day 8 (FIG. 1B), day 15 (FIG. 1C), and day 29 (FIG. 1D), after MCAO. The brains of the stroke rats were cut into seven continuous slices for each group. The damaged area and atrophy are shown by the yellow arrowheads.

FIG. 2A-FIG. 2I are images showing that the transplantation of HUMSCs alleviated the damage range of infarct cortex. FIG. 2A-FIG. 2C show TTC stain images of rat brains at day 8 after the cell transplantation of each group: Group (A)—stroke rats treated with phosphate buffered saline (PBS) (see FIG. 2A); Group (B)—stroke rats that were transplanted with HUMSCs untreated with neuron-conditioned medium (NCM) (see FIG. 2B); and Group (C)—stroke rats that were transplanted with HUMSCs treated with NCM (see FIG. 2C). The damaged area and atrophy are shown by the yellow arrowheads. FIGS. 2D-2F represent the brains of stroke rats evaluated after being fixed with paraformaldedyde at 36 days after cell transplantation in each group: Group (D)—stroke rats treated with PBS (see FIG. 2D); Group (E)—stroke rats transplanted with HUMSCs untreated with NCM (see FIG. 2E); and Group (F)—stroke rats transplanted with HUMSCs treated with NCM (see FIG. 2F). FIGS. 2G-2I represent brain sections stained with cresyl-violet at day 36 after transplantation in each group: Group (G)—stroke rats treated with PBS (see FIG. 2G); Group (H)—stroke rats transplanted with HUMSCs untreated with NCM (see FIG. 2H); and Group (I) stroke rats transplanted with HUMSCs treated with NCM (see FIG. 2I).

FIG. 3A to FIG. 3I are the images of MRI study after MCAO. After MCAO, T2-weighted imaging (T2WI) were acquired in the stroke rats treated with PBS (FIGS. 3A-3E), HUMSCs (FIGS. 3F-3K), and HUMSCs treated with NCM (FIGS. 3L-3Q) continuously at day 1 (A1-A5, F1-F5 and L1-L5), day 8 (B1-B5, G1-G5 and M1-M5), day 15 (C1-C5, H1-H5 and N1-N5), day 22 (D1-D5, I1-I5 and O1-O5), day 29 (E1-E5, J1-J5 and P1-P5) and day 36 (K1-K5 and Q1-Q5) after transplantation. Severe inflammation and edema were expressed at 1 day after transplantation in all three groups (shown by the red arrows). Some atrophy of the damaged cortex was displayed at 8 day after PBS injection in control group (shown by the yellow arrows). At day 29 after transplantation, the infarct cortex almost disappeared in the control group (shown by the green arrows), but were still preserved until day 36 in stem cell and NCM groups (shown by the blue arrows).

FIG. 4A and FIG. 4B are images showing stroke-induced behavior deficits recovered after HUMSCs transplantation. The stroke rats transplanted with untreated HUMSCs or treated with NCM HUMSCs showed better functional recovery in cylinder test (A) (FIG. 4A) and rotarod test (B) (FIG. 4B) than the control group (FIG. 4C). Data are expressed as mean±SEM. * indicates significant difference (p<0.05) compared with control group at the same day. # indicates significant difference (p<0.05) compared with normal value before MCAO in the same group.

FIG. 5 has photomicrographs showing the existence and distribution of HUMSCs in rat brain 36 days after transplantation. The line drawings of rat brain demonstrate the extent of HUMSCs migration after implantation in the infarcted cortex of the rat at Bregma level. Cell migration patterns were followed by bis-benzimide labeling (blue) in serial brain sections.

### DETAILED DESCRIPTION OF THE INVENTION

As used herein the following terms may be used for better interpretation of claims and specification.

The articles “a” and “an” are used herein to refer to one or more than one (i.e., at least one) of the grammatical object of the article. By way of example, “an element” means one element or more than one element.

As used herein, the term “human umbilical mesenchymal stem cell (HUMSC)” refers to cells of the mesenchymal tissue in human umbilical cord (i.e., the so-called Wharton's Jelly). The method of isolation and culturing HUMSCs is described in detail in Example 1.

As used herein, the term “ischemic brain injury” refers to an absolute or relative shortage of the blood supply to the brain, with resultant damage or dysfunction of cerebral tissue, especially central nerve cells. The term “ischemia” as used herein refers to an inadequate or stopped flow of blood to a part of the body, caused by constriction or blockage of the blood vessels supplying it. Ischemic brain injury can be the result of various diseases, or the result of arterial obstruction such as strangulation. Similarly to cerebral hypoxia, severe or prolonged cerebral ischemia will result in unconsciousness, brain damage or death, mediated by the ischemic cascade.

The term “stroke” as used herein refers to a sudden loss of function caused by an abnormality in the blood supply to the brain. Ischemia (diminished or stopped blood flow) and infarction (cell damage and death within the zone of ischemia) are the pathologic processes in stroke that lead to neurologic deficits. Risk factors for stroke include advanced age, hypertension (high blood pressure), previous stroke or transient ischemic attack (TIA), diabetes, high cholesterol, cigarette smoking, atria fibrillation, migraine with aura, and thrombophilia (a tendency to thrombosis). Blood pressure is the most important modifiable risk factor of stroke.

Strokes can be classified into two major categories: ischemic and hemorrhagic. “Ischemic stroke” is caused by obstruction of blood vessels supplying the brain. The primary subcategories of ischemic stroke are thrombotic stroke, embolic stroke and lacunar infarctions. “Hemorrhagic stroke” is caused by the rupture of blood vessels supplying the brain. The primary subcategories of hemorrhagic stroke are subarachnoid hemorrhage (SAH) and intracerebral hemorrhage (ICH).

As used herein, the term “neurological damage” refers to neuronal apoptosis or dysfunction caused by loss of oxygen, head trauma, toxic injuries, infection or inflammation. According to the damage of areas or neuron types, a subject suffering from neurological damage may have paralysis or movement disorder, memory loss, depression or consequent problems. “Treating or preventing an ischemic brain injury or neurological damage” as used herein refers to reversing, curing, healing, relieving, ameliorating, alleviating or stopping the process of ischemic brain injury or neurological damage as aforementioned.

As used herein, a “subject” is any animal subject to ischemic brain injury or neurological damage. In addition to humans, a subject typically includes mammals, such as simians, felines, canines, equines, bovines, porcines, ovines, caprines, murines, mammalian farm animals, mammalian sport animals, mammalian pets and mammalian laboratory animals.

As used herein, a “therapeutically effective amount” of, for instance, cells, with respect to the method of the present invention, refers to an amount of the therapeutic dosage regimen that is effective in treating or preventing an ischemic brain injury or neurological damage. Such a therapeutically effective amount prevents damage to or causes an improvement in neuronal function according to clinically acceptable standards. According to an embodiment of the invention, the amount of HUMSCs transplanted to the area of the injury is about 10⁴ to about 10⁵ cells.

The present invention relates to the use of HUMSCs obtained from Wharton's Jelly in treating or preventing ischemic brain injury. Accordingly, the present invention relates to a method for treating or preventing an ischemic brain injury or neurological damage due to ischemia in a subject comprising transplanting human umbilical mesenchymal stem cells (HUMSCs) obtained from Wharton's Jelly to the ischemic areas of brain injury of said subject. The ischemic brain injury may caused by ischemic stroke, hemorrhage stroke, or a head trauma. In one embodiment, HUMSCs obtained from Wharton's Jelly was used to treat ischemic stroke.

To transplant HUMSCs to the ischemic area of brain injury or neurological damage, the HUMSCs may be directly delivered to an exposed or affected area by means of injection. In a preferred embodiment of the present invention, the HUMSCs are delivered to the desired area by direct injection. The HUMSCs may also be delivered to the ischemic area of brain injury or neurological damage neat or by means of a pharmaceutically acceptable vehicle. As used herein, a “pharmaceutically acceptable carrier” refers to a non-toxic solid, semisolid or liquid filler, diluent, encapsulating material or formulation auxiliary of any conventional type. A “pharmaceutically acceptable vehicle” is non-toxic to a subject at the dosages and concentrations employed, and is compatible with the HUMSCs and any other ingredients of any formulation comprising the HUMSCs. For example, a suitable pharmaceutically acceptable vehicle for a formulation containing the HUMSCs may include, but is not limited to, mannitol, water, Ringer's solution, and isotonic sodium chloride solution. When transplanted or administered to a subject with a pharmaceutically acceptable vehicle, the HUMSCs with the vehicle may be administered by direct injection, arterial or venous infusion.

The amount of HUMSCs to be transplanted to the ischemic area of brain injury varies in view of many parameters, such as the condition of the subject and the type and severity of the brain injury or neurological disorder. The amount of HUMSCs, when applied to the subject suffering from ischemic brain injury or neurological disorder, should attain a desired effect, i.e., inducing neural proliferation, replacing dead nerves or nerve cells, or at least partially functional recovery of the injured neuron. A suitable amount can be readily determined in view of the present disclosure by persons of ordinary skill in the art without undue experimentation. In a preferred embodiment of the present invention, the amount of HUMSCs transplanted to the area of the brain injury is about 10⁴ to about 10⁵ cells, preferably 5×10⁵ cells.

The method of the present invention can also recover neurological behavior deficits from an ischemic brain injury or neurological damage due to ischemia in a subject comprising transplanting human umbilical mesenchymal stem cells (HUMSCs) obtained from Wharton's Jelly to the ischemic areas of brain injury or neurological damage of the subject. If the area of the brain affected involves the cerebellum, the subject may have one, some or all following symptoms: trouble walking, altered movement coordination, vertigo and/or disequilibrium. According to the present invention, transplantation of HUMSCs may induce proliferation of endogenous neural precursor cells, and replace dead cells. As result, the subject's mobility would recover to at least some extent, if not largely or totally.

Without further elaboration, it is believed that one skilled in the art can, based on the above description, utilize the present invention to its fullest extent. The following specific embodiments and examples are, therefore, to be construed as merely illustrative, and not limitative of the remainder of the disclosure in any way whatsoever. All publications cited herein are hereby incorporated by reference herein.

### EXAMPLES

Materials and Methods

Preparation of HUMSCs from Wharton's Jelly

Human umbilical cords were collected in Hanks' balanced salt solution (HBSS) (14185-052, Gibco, Grand Island, N.Y.) at 4° C. After disinfection in 75% ethanol for 30 seconds, the umbilical cord vessels were cleared off while still in HBSS. The mesenchymal tissue (in Wharton's jelly) was then diced into cubes of approximately 0.5 cm³ and centrifuged at 250×g for 5 minutes. After removal of the supernatant fraction, the precipitate (mesenchymal tissue) was washed with serum-free Dulbecco's modified Eagle's medium (DMEM) (12100-046, Gibco) and centrifuged at 250×g for 5 minutes. After aspiration of the supernatant fraction, the precipitate (mesenchymal tissue) was treated with collagenase at 37° C. for 18 hours, washed, and further digested with 2.5% trypsin (15090-046, Gibco) at 37° C. for 30 minutes. Fetal bovine serum (FBS) (SH30071.03, Hyclone, Logan, Utah) was then added to the mesenchymal tissue to neutralize the excess trypsin. The dissociated mesenchymal cells were further dispersed by treatment with 10% FBS-DMEM and counted under a microscope with the aid of a hemocytometer. The mesenchymal cells were then used directly for cultures or stored in liquid nitrogen for later use.

Preparation of Neuronal Conditioned Medium

Seven-day postnatal Sprague-Dawley rats were anesthetized by pentobarbital. The brains were removed, placed in Ca²⁺/Mg²⁺-free buffer (14185-052, Gibco), and centrifuged at 1000×g for 5 minutes. After removal of the supernatant fraction, 10% FBS-DMEM was added to the precipitate (brain tissue). The brain tissue suspension was triturated 15 times for dispersal into single cells. The cells were suspended in 10% FBS-DMEM and incubated at 37° C. in 5% CO₂ and 95% O₂. To inhibit the growth of glial cells, 2 μM of 1-(β-D-Arabino-furanosyl)-cytosine hydrochloride (AraC) (c-6645, Sigma-Aldrich, St. Louis, Mo.) was added on the next day. On the fifth day of culture, the culture medium was removed. This is called neuronal-conditioned medium (NCM) to be used for the culture of umbilical mesenchymal cells. The HUMSCs were cultured in NCM alone, which was replaced every other day.

Preparation of Stroke Animals (Medial Cerebral Artery Occlusion (MCAO) Surgery)

Adult Sprague-Dawley rats (280 to 360 g) were used in this study. Under chloral hydrate anesthesia (400 mg/kg i.p.), the rats were placed in a stereotaxic frame. To induce cerebral infarction, ligations of the right middle cerebral artery (MCA) and bilateral common carotid arteries (CCAs) were performed by methods described previously (Lin, T., et al., Stroke 1993, 24: 117-121). With the use of a surgical microscope, the animal was placed in the lateral position, and a curved vertical 1-cm skin incision was made just by the right orbit. Splitting of the temporalis muscle, a 3-mm² burr hole was drilled at the junction of the zymotic arch and the squamous bone. The MCA was exposed and ligated with 10-0 suturing. Immediately, both common carotid arteries were than occluded using nontraumatic aneurysm clips. After a duration of ischemia for 90 minutes, blood flow was restored in all three arteries.

Experimental Grouping

24 hours after MCAO, the Sprague-Dawley rats were divided into three groups: (1) control group: the rats received PBS into cerebral cortex; (2) stem cell group: the rats received a suspension of 5×10⁵ graft HUMSCs; and (3) NCM 6D group: the rats received a suspension of 5×10⁵ graft HUMSCs that had been cultured in NCM for 6 days.

Preparation and Transplantation of HUMSCs

HUMSCs untreated or treated with NCM for 6 days were trypsinized at 37° C. for 5 minutes with 0.25% trypsin, and the dissociated cells were resuspended in PBS. A total of 5×10⁵ cells separated into two injections for two different positions in the infarcted cortex of each rat: (1) anteroposterior=+1.2 mm, lateral=+5.2 mm, ventral=−4.0 mm and (2) anteroposterior=−2.8 mm, lateral=+6.2 mm, ventral=−5.0 mm, based on positioning from the Bregma and skull surface. The control group only received PBS. A waiting period of 10 minutes before the needle was removed allowed the cells to settle. The rat hosts did not receive any immunosuppression medications.

To inspect distribution of HUMSCs transplantation in the ischemic cortex, HUMSCs before transplantation were treated with 1 μg/ml bis-benzimide (B2883, Sigma-Aldrich) for 24 hours to label the cells.

TTC Staining

At day 1, 8, 15 and 29 after MCAO, experimental rats were deeply anaesthetized with chloral hydrate (400 mg/kg i.p.) and decapitated. The brains were removed carefully and dissected into coronal 2-mm-thick sections using a brain slicer. The fresh brain slices were immersed in a 2% solution of 2,3,5-triphenyltetrazolium chloride (TTC) (Sigma) in normal saline for 30 min. The stained slices were fixed with 10% of formalin at 4° C. Brain slices lacking red staining defined the infarct area. The images of slices were acquired with a scanner and analyzed using Image-Pro™ software (Media Cybernetics, Inc.).

Cresyl-Violet Staining

Brain tissue sections were stained in 1% crystal violet solution for 10 minutes and followed by dehydration and mounting. Pathological changes and variations were observed under an optical microscope.

MRI Study and Measurement of Infarct Volume

Experimental animals were imaged at day 1, 8, 15, 22, 29, and 36 after transplantation using high resolution 3-Tesla MRI system (Biospec, Bruker Companies, Ettlingen, Germany). T2-weighted imaging (T2WI) of the whole brain was acquired from each rat. The pulse sequences were obtained with the use of a spin-echo technique (repetition time, 3500 ms; echo time, 62 ms). Under anesthesia, 20 coronal and transverse image slices 1-mm thick were scanned without any gaps. These image slices were analyzed using Image-Pro™ software. The atrophy volume of damaged cortex was that total cortical volume of the left hemisphere subtracted the noninfarcted volume in the right cortex.

Behavioral Test

Two different kinds of behavioral tests were used in the study, performed before and after MCAO, and at day 1, 4, 8, 15, 22, 29, and 36 after transplantation, the cylinder test and the rotarod test. The cylinder test, detecting forelimb asymmetry, was examined by placing the rats in a transparent cylinder 20-cm in diameter and 30-cm high for 3 minutes (Schallert, T., et al., Neuropharmacology 2000, 39: 777-787). Rats in the cylinder were encouraged to vertically explore the walls with forelimbs, but the walls were high enough so that the rats could not reach the top. The experimenter videotaped the rat's activity from a ventral view. Forelimb use was estimated during vertical exploration. Each forepaw contact with the cylinder wall was counted. The asymmetry score of forelimb use in wall exploration was calculated as the percent preference for the paw ipsilateral or contralateral to the lesion used for reaching: preference=ipsilateral paw/(ipsilateral paw+contralateral paw)×100 (Schallert, T., et al., supra).

For the rotarod test, rats were placed on an accelerating rotating cylinder (the rotarod), and the duration of time the animals remained on the rotarod was measured. The speed was slowly increased from 4 to 40 rpm within 5 minutes. A trial ended if the animal fell off the rotarod or gripped the device. The rats were trained 3 days before MCAO. The mean duration (in seconds) on the device was recorded with 3 rotarod measurements 1 day before surgery. Data are presented as percentage of mean durations (3 trials) on the rotarod after surgery and treatment compared with internal baseline control (before surgery) (Chen, J., et al., Stroke 2001, 32: 1005-1011).

Histological Examination and Immunochemical Analysis of Grafted Brain Cryosections

For tracking the transplanted cells, the cellular membrane penetrating and DNA-binding fluorescence probe bis-benzimide was used. Thirty-six days after transplantation, the grafted rats were anesthetized terminally using an overdose of chloral hydrate i.p. Rat brains were fixed by transcardial perfusion with saline, followed by perfusion and immersion in 4% paraformaldehyde in PBS for 24 hours at 4° C. Next, the specimens were equilibrated in 30% sucrose in PBS for 4-5 days at 4° C., then embedded in optimal cutting temperature (OCT) compound and frozen at −20° C. Sections were cut into serial 30-μm thick slices using a cryostat. The tissues were stained with the fluorescent stain bis-benzimide and visualized under a fluorescence microscope for mapping of the stained cells.

Results

### Example 1

### The Infarct Volume of Stroke Rats After MCAO Surgery

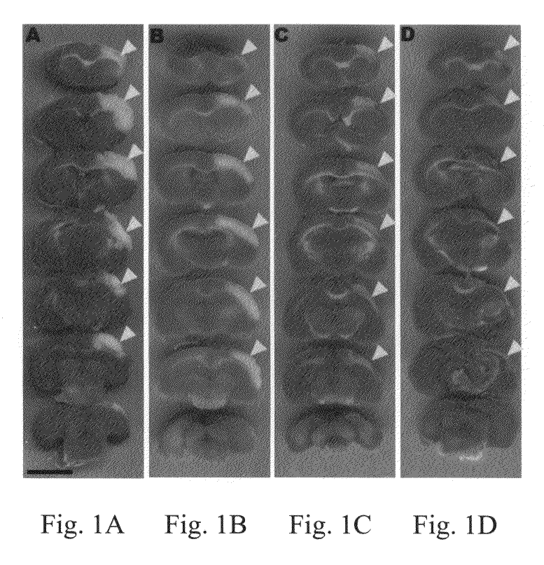

Using the histological examination and immunochemical analysis of grafted brain cryosections explained above, the ranges of damaged cortex in stroke rats were examined. Individual groups of stroke rats were sacrificed at day 1, 8, 15, or 29 after MCAO surgery. These rats were used for TTC stain, a common method in stroke rodent study (Bederson, J. B., et al., Stroke 1986, 17: 1304-1308). Normal brain tissue was stained for red color, and damaged areas appeared as white color. FIG. 1A showed that quantitative analysis of infarcted brain volume demonstrated that the damaged cortex was inflamed and edemic, resulting in a significant increase of volume at 1 day after MCAO (p<0.05; 212.70±7.55 mm³). At day 8, as shown in FIG. 1B, after MCAO, the edema of the inflamed cortex was alleviated, and the volume of damaged brain was 154.29±6.52 mm³. FIG. 1C showed that the infarcted cortex started to express atrophy compared with the contralateral normal cortex at day 15 after MCAO. At day 29 after MCAO, FIG. 1D showed that the infarcted cortex almost displayed total atrophy, and the degenerative volume was 163.56±12.64 mm³. These results indicated the changes of infarct cortex in the stroke brains after MCAO.

### Example 2

### HUMSCs Transplantation Reduces Damage Area of Stroke Rat Brain

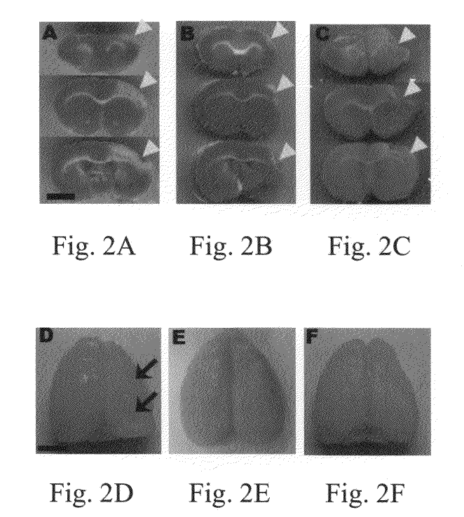

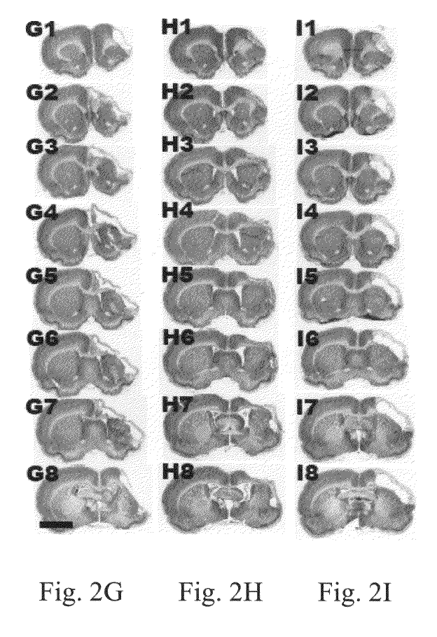

At day 8 after transplantation, the changes of ischemic cortices using TTC stain were examined for three different groups (Control, Stem cell, and NCM 6D). FIGS. 2A-2C illustrated that the damaged areas of stroke rat brains in the stem cell group and the NCM 6D group are significantly reduced as compared with the control group. At day 36 after transplantation, representative brain cortical expression from stroke rats in each of the control, stem cell and NCM 6D groups is shown in FIGS. 2D-2F, respectively. The appearance of atrophy in the control group was more serious in the control group (FIG. 2D) than in the stem cell (FIG. 2E) and the NCM 6D (FIG. 2F) groups. FIGS. 2G-2I showed the similar patterns from rostral to caudal slices of brain stained with cresyl-violet stain. The brain slices shown in FIG. 2G1-2G8 from the control group displayed harsh damage in the infarct cortex and even in the basal ganglion.

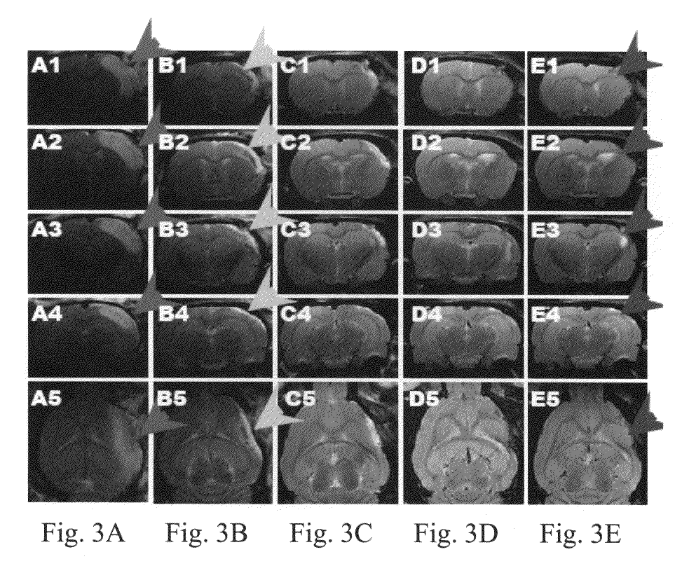

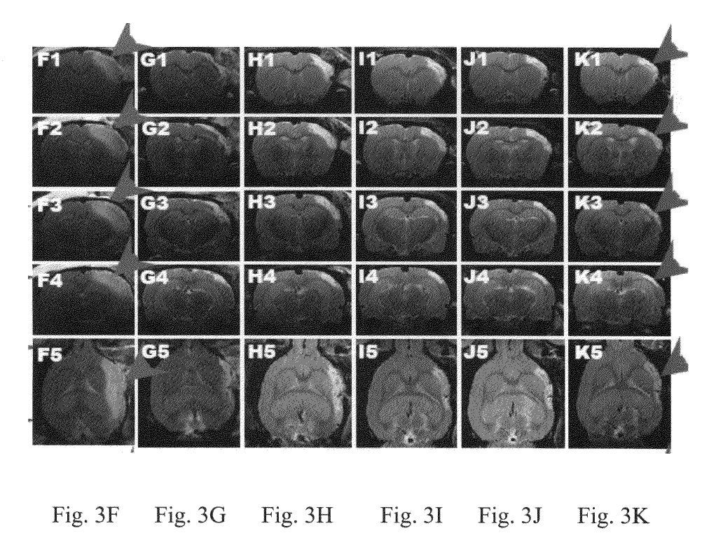

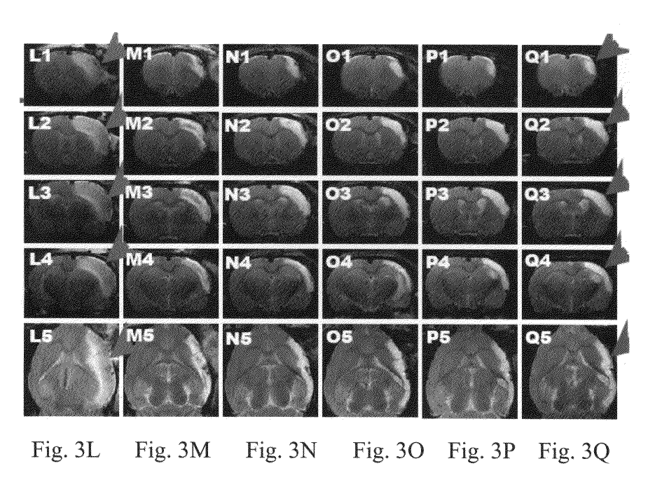

In order to observe the infarct volume of the same rat, using MRI was necessary (FIG. 3). The MRI results showed that the ischemic cortex displayed inflammation and edema in the PBS group at day 1 (FIGS. 3A1-3A5, 287.33±6.34 mm³). From 8 to 29 days after PBS injection in control group, atrophy of the infarct volume changed from 30.04±7.63 mm³ to 111.47±5.43 mm³ (FIGS. 3B1-3B5, 3C1-3C5, 3D1-3D5, and 3E1-3E5). The ischemic cortex almost disappeared 29 days after PBS injection. However, in the stem cell and NCM 6D groups, the ischemic cortex also displayed inflammation and edema at 1 day after transplantation of HUMSCs that were untreated or treated with NCM and the infarct volumes were 286.61±36.79 mm³ and 332.86±30.11 mm³ (FIGS. 3F1-3F5 and 3L1-3L5). In all three groups, the atrophy volumes on days 15, 22 and 29 were greater than at day 8. But especially in the control group, the atrophy volume at day 29 was even greater than at day 15 (as compared to FIGS. 3C1-3C5 and FIGS. 3E1-3E5). This expression was not shown in the stem cell and NCM 6D groups, and the changes of atrophy volume were mild from day 15 to day 29 (FIGS. 3H-3J and 3N-3P). Furthermore, most infarct cortex could also be preserved in these cell transplantation groups until day 36 (FIGS. 3K1-3K5 and 3Q1-3Q5). From day 8 to 29 after cell transplantation, the atrophy volume of the infarct cortex in the stem cell and NCM 6D groups significantly decreased in comparison to the volume of the control group at the same day. In summary, the injury volumes (edema volume plus atrophy volume) were not different in these three groups, but transplantation of HUMSCs (untreated or treated with NCM) prevented the infarct cortex from disappearing, because transplanted HUMSCs would differentiate into neural cells to replace the dead ones in the infarct cortex.

### Example 3

### HUMSCs Transplantation Recovers Neurological Behavior Deficits

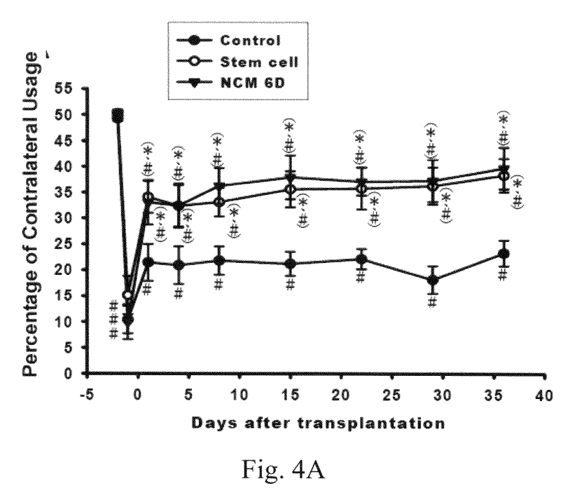

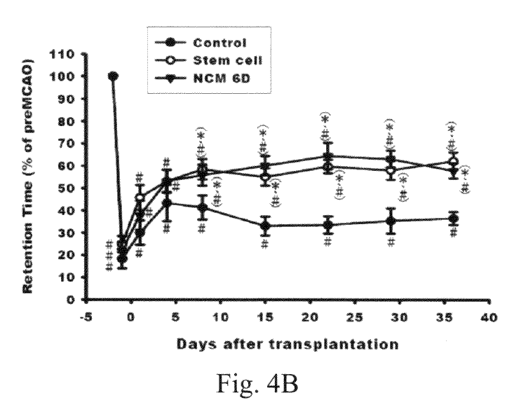

The effects of HUMSCs transplantation on functional recovery were examined for the cylinder and rotarod tests. These tests were performed before and after MCAO, and at days 1, 4, 8, 15, 22, 29, and 36 after transplantation. In the cylinder test as shown in FIG. 4A, normal rats before MCAO used both forelimbs at almost same percentage, displayed as 50%. After MCAO (day −1), contralateral forelimb usage percentage of the control group treated without stem cells decreased to 10.38±2.65%, and increased to 21.44±3.55% after treatment with PBS. But compared to the normal value, the tendency still showed a statistically significant difference until 36 days (p<0.05, as shown in FIG. 4A). In the stem cell group and NCM 6D group, the percentage of contralateral forelimb usage was decreased after MCAO (15.13±3.71% and 9.94±3.37%, respectively) and differed from the normal value (p<0.05, as shown in FIG. 4A). One day after transplantation of stem cells without and with NCM treatment, the percentage of contralateral forelimb usages was raised to 34.09±3.09% in the stem cell group and to 33.05±4.30% in the NCM 6D group, but still differed from the normal value (p<0.05, as shown in FIG. 4A). This tendency was sustained until 36 days, just as the control group did. As shown by the data, one day after MCAO, usage of contralateral forelimb decreased to 19.84%, about 30.13% of the normal value. However, usage of the contralateral forelimb recovered to 65.95%, about 67.88% of the normal value after transplantation in the stem cell group and the NCM 6D group. The difference compared with the control group was significant (p<0.05, as shown in FIG. 4A).

In the rotarod test, the sustainable time of every rat on the accelerating roller was measured and recorded as the normal baseline before MCAO. One day after MCAO, the sustainable time in the control group decreased to 18.23±4.17% of the normal baseline, and increased to 29.94±5.45% one day after PBS injection (FIG. 4B). However there was still a statistical decrease as compared with the normal value until 36 days (p<0.05, as shown in FIG. 4B). In the stem cell group and NCM 6D group, the sustainable times were decreased to 24.86±3.58% and 22.05±3.72%, respectively, of the normal baseline (FIG. 4B). At day 8 after transplantation of the stem cell groups without with treatment with NCM, the sustainable times were increased to 58.46±4.61% and 55.88±4.69%, respectively, and differed from the control group significantly (p<0.05, FIG. 4B). This tendency was maintained for at least 36 days. These results indicated that the rats treated with transplantation with HUMSCs (untreated or treated with NCM) recovered motor functional deficits caused by MCAO.

### Example 5

### HUMSCs Can Survive and Migrate in Stroke Rat Brain

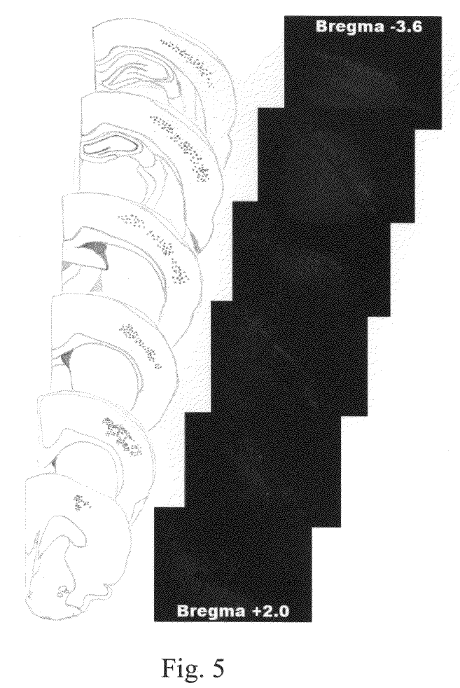

At day 36 after transplantation of HUMSCs, cell migration patterns were followed by bis-benzimide labeling in 30-μm serial sections. The labeled cells had migrated in both directions of the rostrocaudal axis from the two implantation sites (Bregma +1.2 and −2.8). As shown in FIG. 5, most of the labeled cells were localized in the region of Bregma +2.0 to the region of Bregma −3.6, throughout most of the infarcted cortex.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims

What is claimed is:

1. A method for treating or preventing an ischemic brain injury or neurological damage due to ischemia in a subject, the method comprising transplanting a therapeutically effective amount of human umbilical mesenchymal stem cells (HUMSCs) obtained from Wharton's Jelly to the ischemic areas of the brain injury or the neurological damage of the subject.

2. The method according to claim 1, wherein the transplantation of HUMSCs is achieved by direct injection of HUMSCs to the areas of ischemic brain injury or neurological damage.

3. The method according to claim 1, wherein transplantation of the HUMSCs prevents atrophy of infarct cortex of the subject.

4. The method according to claim 1, wherein the ischemic brain injury or neurological damage is caused by ischemic stroke, hemorrhage stroke or a head trauma.

5. The method according to claim 1, wherein the ischemic brain injury or neurological damage is caused by ischemic stroke.

6. The method according to claim 5, wherein the ischemic stroke is caused by thrombosis, embolism, systemic hypoperfusion, or venous thrombosis.

7. A method for recovering neurological behavior deficits from an ischemic brain injury or neurological damage due to ischemia in a subject, the method comprising transplanting human umbilical mesenchymal stem cells (HUMSCs) obtained from Wharton's Jelly to the ischemic areas of brain injury or neurological damage of the subject.

8. The method according to claim 7, wherein the transplantation of HUMSCs is achieved by direct injection of HUMSCs to the areas of ischemic brain injury or neurological damage.

9. The method according to claim 7, wherein the ischemic brain injury or neurological damage is caused by ischemic stroke, hemorrhage stroke, or a head trauma.

10. The method according to claim 7, wherein the ischemic brain injury or neurological damage is caused ischemic stroke.

11. The method according to claim 10, wherein the ischemic stroke is caused by thrombosis, embolism, systemic hypoperfusion, or venous thrombosis.
